(19) United States
(12) Patent Application Publication
(10) Pub. No.: US 2007/0260479 A1
(43) Pub. Date: Nov. 8, 2007

(54) Method and system for consolidating diverse clinical data to facilitate medication titration

(76) Inventor: David Duckert, Menomonee Falls, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/416,433

(22) Filed: May 2, 2006

(51) Int. Cl. G06Q 10/00; A61B 5/00

(52) U.S. Cl. 705/2; 600/300

(57) ABSTRACT

The method and system includes patient sensing systems configured to collect a plurality of physiological data sets and treatment data sets from a patient. A user selects an information set to be viewed such as a pre-defined condition or common medication combination. The method and system constructs a trended view of the information set over a period of time, wherein the trended view includes a pre-defined combination of the plurality of physiological and treatment data sets for a particular information set. The trended view can then be displayed to assist the user with medication titration.

### FIELD OF THE INVENTION

The invention relates to the field of patient monitoring. More particularly, the invention relates to the field of chronic disease monitoring.

### BACKGROUND OF THE INVENTION

When treating patients with congestive heart failure (CHF), a common suite of medications are often prescribed. These medications generally include: diuretics, used to increase urinary output to counteract edema, e.g., Lasix; beta-blockers, used to lower heart rate and blood pressure, e.g., Carvedilol; ACE Inhibitors used to lower arteriolar resistance and increase venous capacitance, increase cardiac output, stroke volume, and lower renovascular resistance, e.g., Rampiril; and anticoagulant, used to thin blood to reduce clotting, e.g., Coumadin. Determining the correct dose of these medications is difficult. Some of these drugs work in concert, and others interact with each other. For some, the dose may need to be slowly increased to reach an effective level. At higher levels, these medications may cause undesirable side-effects such as cough, rash, or renal impairment. In practice, titrating these drugs is a very complex process.

To effectively titrate these medications, a variety of information is required. Historical information regarding dosages, side effects, lab results and physiological values should be considered. Often this information is not readily available, or if it is available, it is not consolidated into a single view. This makes it very difficult and time consuming for clinicians to make adjustments to medications.

As an example, a clinician is trying to control edema using a diuretic. The main feedback method used to titrate the dosing level is the patient's weight. An optimal or “dry” weight is known. A starting dose of a diuretic perhaps 100 mg of Lasix is prescribed. After a period of time the patient's weight is still not controlled and periods of dyspnea are being reported by the patient. The clinician would like to increase the dose of Lasix, but must first determine: The current dose; if this higher dose has been unsuccessfully tried in the past, or if this higher dose would exceed the maximum dose. After searching the medical record of the patient to gather the data, the dose is increased to 300 mg. The effect of some blood pressure medications can be increased with Lasix, so the clinician makes a note to monitor the patient's blood pressure closely.

A few weeks later the patient's weight now seems to be controlled and the episodes of dyspnea are eliminated, but the patient now complains of hearing loss and rash. Seeing the note in the file, the patient's last blood pressure value is located and reviewed, and seems a bit high. The clinician tries to figure out whether this reading was taken before or after the change in dose. The clinician would like to decrease the dose of Lasix to reduce the side effects, but must first determine if this lower dose has been tried in the past or if any other med changes have been made recently that could be responsible for the reported side effects. After searching the medical record of the patient, it is determined that a much lower dose (100 mg) was ineffective in the past. The clinician hypothesizes that a dose of 200 mg might still be effective and but yet not cause the side effects. The clinician prescribes a dose of 200 mg.

This very simple case illustrates the process clinicians perform to titrate a diuretic. In a real case, drug interactions would come into play and lab results for renal function would also be considered. Unfortunately, much of the information the clinician needs to make these decisions is not consolidated or presented in a meaningful way. The clinician must first collect the information from a variety of different systems and then construct a mental historical timeline of dosages, side effects and lab results.

### SUMMARY OF THE INVENTION

The method and system includes patient sensing systems configured to collect a plurality of physiological data sets and treatment data sets from a patient. A user selects an information set to be viewed such as a pre-defined condition or common medication combination. The method and system constructs a trended view of the information set over a period of time, wherein the trended view includes a pre-defined combination of the plurality of physiological and treatment data sets for a particular information set. The trended view can then be displayed to assist the user with medication titration.

In one aspect of the present invention, a method of facilitating medication titration comprises collecting a plurality of physiological data sets and treatment data sets from a patient, selecting an information set to be viewed and constructing a trended view of the information set, wherein the trended view includes a pre-defined combination of the plurality of physiological and treatment data sets for a particular information set that is based on a particular medical condition and wherein the information set is based on a common drug mixture. The method further comprises displaying a trended view on a graphical user interface, administering a treatment action in response to the trended view which includes administering a medication and ordering a lab analysis.

Another aspect of the present invention is a system for facilitating medication titration comprising a monitoring system configured to collect a plurality of physiological data sets and treatment data sets from a patient, a storage media for storing a computer application, and a processing unit coupled to the monitoring system and the storage media, and configured to execute the computer application, and further configured to receive the plurality of physiological data sets and treatment data sets from the monitoring system, wherein when the computer application is executed and a user selects an information set to be viewed, a trended view of the information is constructed and displayed on a graphical user interface, wherein the trended view includes a pre-defined combination of the plurality of physiological and treatment data sets for a particular information set. A system user administers treatment action in response to the trended view wherein the treatment action includes administering a medication and ordering lab analysis and wherein the information set is based on a particular medical condition and further wherein the information set is based on a common drug mixture.

Yet another aspect of the present invention is a method of constructing a trended view for facilitating medication titration, the method comprising collecting a plurality of physiological data sets and treatment data sets from a patient, selecting an information set to be viewed, configuring a pre-defined combination of the plurality of physiological and treatment data sets for a particular information set in the trended view, and displaying the trended view on a graphical user interface such that a user may view the trended view and administer a treatment action to the patient in response to the trended view.

### BRIEF DESCRIPTION OF THE DRAWINGS

FIG. 1 illustrates a flow chart of a method in accordance with an embodiment of the present invention.

FIG. 2 illustrates a graphical representation of a graphical user interface in accordance with an embodiment of the present invention.

FIG. 3 illustrates a block diagram of a system in accordance with an embodiment of the present invention.

### DETAILED DESCRIPTION OF THE INVENTION

The facilitation method and system of the present invention collects diverse information from a variety of systems such as an electronic medical record, a pharmacy record, a home monitoring system, and creates a trended view of the diverse information for that patient. The method and system allow a user to construct specific views defined for various conditions such as congestive heart failure, diabetes, and others, as well as for pre-defined combinations of drugs that are commonly used in treating patients. The method and system collects this information and displays it in a trended view over a period of time such that a physician has a complete look at a patient's physiological response to the treatment being received from the physician through medication. An example of such a trended view may include a graphic display of he patient's vital signs over a period of time such as weight, creatinine level, or the patient's physical symptoms. The trended view would also include a graphic display of the dosages of the various medications administered to the patient over that same period of time. The method and system will provide the physician with a powerful diagnostic tool that will reduce errors and save the physician a tremendous amount of time.

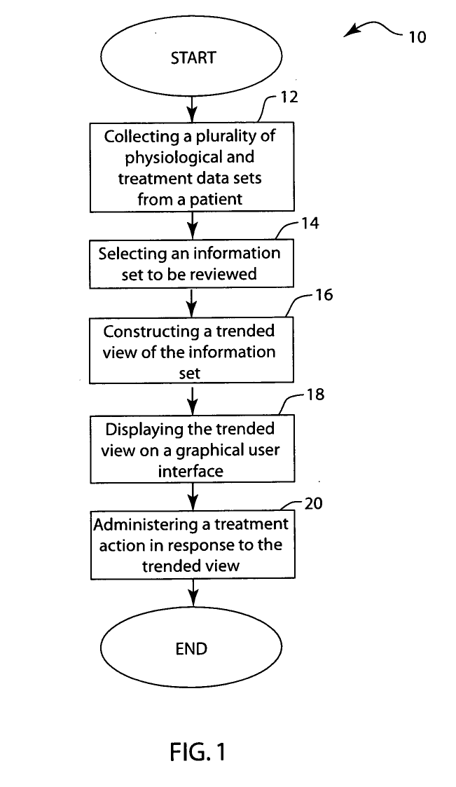

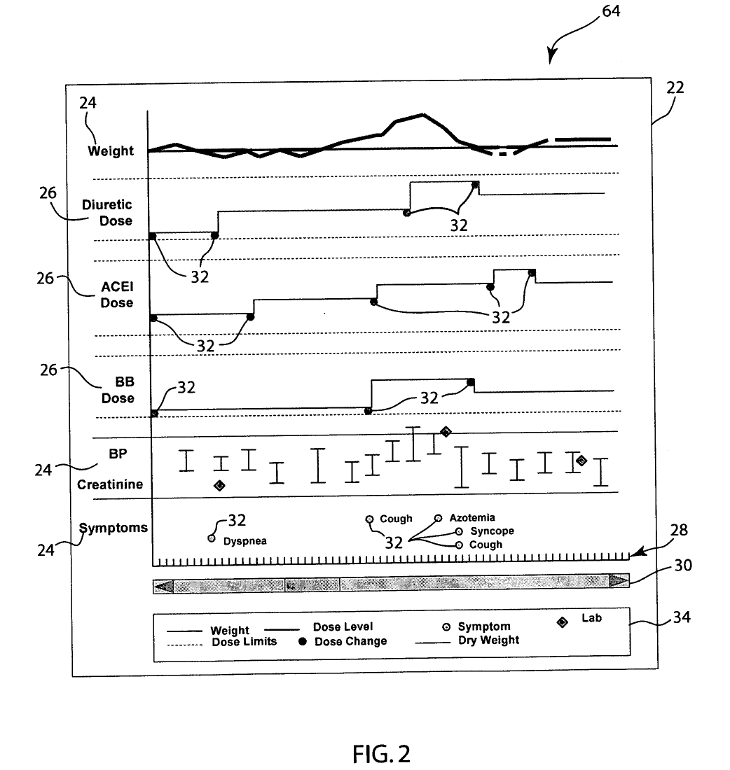

FIG. 1 illustrates an embodiment of such a facilitation method 10. In step 12, a plurality of physiological and treatment data sets are collected from a patient. Examples of such physiological data sets include weight, blood pressure, heart rate, physical symptoms, medication side-effects, or the results of laboratory tests. It should be noted that this list of physiological data sets is not exhaustive and is only an example of the type of data represented by these physiological data sets. The treatment data sets are records of treatment administered to the patient over a period of time that includes administered medication and dosages, as well as any other treatments that may be administered to a patient by the physician. In both the case of the physiological data set as well as the treatment data sets that are collected from a patient, the type and number of such data sets are predetermined according to what the particular patient is being monitored for. An example will be discussed in further detail below and depicted in FIG. 2.

Referring back to FIG. 1, in step 14, an information set to be reviewed is selected. In other words, the physician or other medical professional will select which condition, and therefore which physiological and treatment data sets will be monitored for the patient preferably from a list of information sets that are pre-programmed into the system. Once the information set to be review is selected, in step 16, a trended view of the information set is constructed. Here, the collected physiological data sets as well as the treatment data sets from step 12 are graphically grouped together over a pre-selected period of time. In step 18, the trended view is displayed on a graphical user interface. By displaying this trended view, a physician is able to accurately and quickly determine the effects of the administered medication on the physiological state of the patient, and further administer medications to treat the patients, therefore, in step 20, a treatment action in response to the trended view is administered to the patient.

A graphical user interface 64 is graphically depicted in FIG. 2. Here, the trended view 22 includes a number of physiological data displays 24, as well as a number of treatment data displays 26. Each of the data displays 24, 26 further includes data plots 32 which indicate the level of that particular data at a particular time. A time scale 28 shows the period for which the data sets were collected and a time adjustment bar 30 allows a user to move forward or backward along the time scale 28. A key 34 provides the user with definitions for each of the items in the trended view 22. Still referring to FIG. 2, it should be noted that the graphical user interface 64 may be included in any suitable patient monitoring system. For example, the graphical user interface may be implemented on a pre-existing patient monitor, or implemented as a stand-alone or remote PC or laptop, or even be implemented on a hand-held electronic device that the physician or medical professional carries with him or her. It should also be noted that the graphical user interface may include touch screen capabilities in order to select the appropriate information set to be monitored and displayed, and may further include an input device such as a keyboard, or keypad to further allow the physician or medical professional to indicate the pre-defined information set for viewing. Such input devices would also allow a physician or medical professional to add or delete particular physiological data displays 24 or treatment data displays 26 as desired.

Referring back to the example in the background of the present invention, including the clinician trying to control edema using a diuretic, the physician would have the patient's weight, administered lasix record, as well as the patient's blood pressure records over a period of time at his or her finger tips. The physician would also be able to see the patient's physical symptoms over that same period of time, thus allowing the patient to appropriately administer new medication without reviewing the patient's entire file, thus saving the physician a tremendous amount of time and greatly reducing the chance of the physician making a mistake by missing some vital information. In essence, the graphical user interface 64 in FIG. 2 constructs a mental historical time line of dosages, side effects and lab results to assist the physician in treating the patient properly.

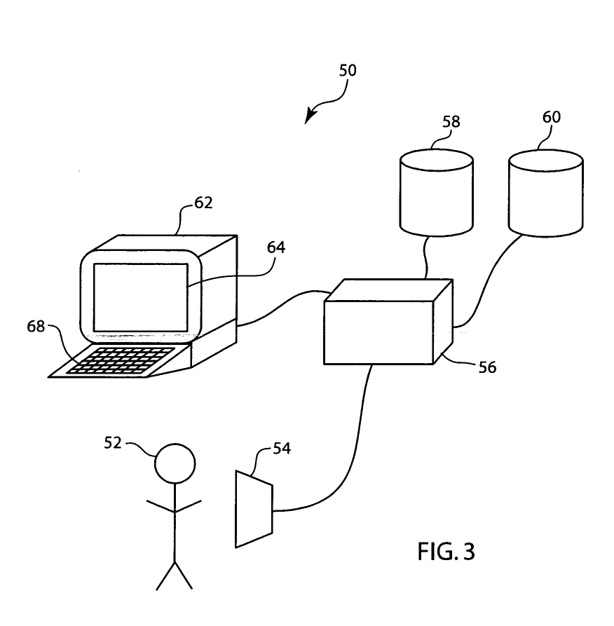

FIG. 3 illustrates an exemplary facilitation system 50 of the present invention. Referring to FIG. 3, a monitoring system 54 collects the appropriate physiological data from the patient 52. As described above, the monitoring system 54 may be any device or system utilized to collect the appropriate information from the patient 52. The monitoring system 54 may include simply a scale, or even a home-monitoring device, or a blood pressure cuff. Furthermore, a database 60 may be accessed to obtain physiological data associated with the patient 52. Such database 60 may include an electronic medical record or the patient's pharmacy record. This method may be implemented in the system as a computer software application, wherein the computer software application is stored on the storage medium 58, and a processor 56 executes the computer software application saved on the storage medium 58, in order to execute the method. The processor 56 will collect the physiological data from the monitoring system 54 and the database 60 and construct the trended view desired by the physician, as received from the input device 68 via the monitoring device 62. As described above, the monitoring device 62 may be an existing patient monitor, a PC or laptop workstation, or even a handheld device by the physician. This monitoring device 62, in some embodiments, will include the input device 68 to allow the physician to select the appropriate information set for the desired trended view, or may be a specialized monitoring device 62 without the input device 68, that would display a specific trended view, such as a specific trended view for congestive heart failure. The trended view will be displayed on the graphical user interface 64, and will be configured such that the physician may modify the trended view with the input device, and further be able to adjust the trended view to examine particular periods of time.

The above described method and system benefits the clinician as it saves time and reduces errors in the administration of drugs to a patient. The method and system collects and presents relevant patient physiological and treatment data in order to make a clinician's decisions about medication dose changes easier.

The present invention has been described in terms of specific embodiments incorporating details to facilitate the understanding of the principals of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

What is claimed is:

1. A method of facilitating medication titration, the method comprising:
collecting a plurality of physiological data sets and treatment data sets from a patient;
selecting an information set to be viewed; and
constructing a trended view of the information set, wherein the trended view includes a pre-defined combination of the plurality of physiological and treatment data sets for a particular information set.

2. The method as claimed in claim 1, further comprising displaying a trended view on a graphical user interface.

3. The method as claimed in claim 1, further comprising administering a treatment action in response to the trended view.

4. The method as claimed in claim 3, wherein the treatment action includes administering a medication.

5. The method as claimed in claim 3, wherein the treatment action includes ordering a lab analysis.

6. The method as claimed in claim 1, wherein the information set is based on a particular medical condition.

7. The method as claimed in claim 1, wherein the information set is based on a common drug mixture.

8. A system for facilitating medication titration, the method comprising:
a monitoring system configured to collect a plurality of physiological data sets and treatment data sets from a patient;
a storage media for storing a computer application; and
a processing unit coupled to the monitoring system and the storage media, and configured to execute the computer application, and further configured to receive the plurality of physiological data sets and treatment data sets from the monitoring system,
wherein when the computer application is executed and a user selects an information set to be viewed, a trended view of the information is constructed and displayed on a graphical user interface, wherein the trended view includes a pre-defined combination of the plurality of physiological and treatment data sets for a particular information set.

9. The system as claimed in claim 8 wherein the user administers treatment action in response to the trended view.

10. The system as claimed in claim 9, wherein the treatment action includes administering a medication.

11. The system as claimed in claim 9, wherein the treatment action includes ordering lab analysis.

12. The system as claimed in claim 8, wherein the information set is based on a particular medical condition.

13. The system as claimed in claim 8, wherein the information set is based on a common drug mixture.

14. A method of constructing a trended view for facilitating medication titration, the method comprising:
collecting a plurality of physiological data sets and treatment data sets from a patient;
selecting an information set to be views;
configuring a pre-defined combination of the plurality of physiological and treatment data sets for a particular information set in the trended view; and
displaying the trended view on a graphical user interface such that a user may view the trended view and administer a treatment action to the patient in response to the trended view.
